(19) United States
(12) Patent Application Publication
(10) Pub. No.: US 2026/0060923 A1
(43) Pub. Date: Mar. 5, 2026

(54) ORAL THIN FILM CONTAINING SEMAGLUTIDE

(71) Applicant: Cure Pharmaceutical, Inc., Oxnard, CA (US)

(72) Inventors: Jose Bernardo, Oxnard, CA (US); Robert Davidson, Woodland Hills, CA (US)

(21) Appl. No.: 19/314,668

(22) Filed: Aug. 29, 2025

Related U.S. Application Data
(60) Provisional application No. 63/688,666, filed on Aug. 29, 2024.

(51) Int. Cl. A61K 9/00; A61K 38/26; A61K 47/10; A61K 47/26; A61K 47/32; A61K 47/38; A61K 47/44

(52) U.S. Cl. CPC A61K 9/006; A61K 38/26; A61K 47/10; A61K 47/26; A61K 47/32; A61K 47/38; A61K 47/44

(57) ABSTRACT

An oral thin film (OTF) containing semaglutide, methods of manufacturing the same, and methods or orally administering the same.

### CROSS-REFERENCE TO RELATED APPLICATIONS

This application claims the benefit of priority to U.S. Provisional Application No. 63/688,666, filed Aug. 29, 2024, the contents of which is hereby incorporated by reference herein.

### BACKGROUND

Semaglutide is an antidiabetic medication used for the treatment of type 2 diabetes and an anti-obesity medication used for long-term weight management.^{[1][2][3]} It is a peptide similar to the hormone glucagon-like peptide-1 (GLP-1), modified with a side chain._{[4][5]} It can be administered by subcutaneous injection or taken orally.^{[6][7][8][9]} It is sold under the brand names Ozempic®^{[7]} and Rybelsus®^{[14]} for diabetes, and under the brand name Wegovy® for weight loss.^{[9]}

Semaglutide was approved for medical use in the US in 2017.^{[7][10]} In 2021, it was the 90^{th} most commonly prescribed medication in the United States, with more than 8 million prescriptions.^{[11][12]}

Semaglutide is indicated as an adjunct to diet and exercise to improve glycemic control in adults with type 2 diabetes.^{[7][8]}

The higher-dose formulation of semaglutide is indicated as an adjunct to diet and exercise for long-term weight management in adults with obesity (initial body mass index (BMI)≥30 kg/m²) or who are overweight (initial BMI≥27 kg/m²) and have at least one weight-related comorbidity.^{[9][13][14]}

In March 2024, the US Food and Drug Administration (FDA) expanded the indication for semaglutide (Wegovy®), in combination with a reduced calorie diet and increased physical activity, to reduce the risk of cardiovascular death, heart attack and stroke in obese or overweight adults with cardiovascular disease.^{[15]}

Semaglutide and similar drugs, such as dulaglutide and liraglutide, have been used to treat binge eating disorder (BED), as they can successfully minimize obsessive thoughts about food and binging urges.^{[16][17]} Some users of these drugs have reported significant reduction in what is colloquially known as “food noise” (constant, unstoppable thoughts about eating despite not being physically hungry), which can be a factor of BED.^{[18][19]}

### REFERENCES

[1] Singh G, Krauthamer M, Bjalme-Evans M (January 2022). “Wegovy (semaglutide): a new weight loss drug for chronic weight management”. Journal of Investigative Medicine. 70 (1): 5-13. doi:10.1136/jim-2021-001952. PMC 8717485. PMID 34706925.
[2] Phillips A, Clements JN (February 2022). “Clinical review of subcutaneous semaglutide for obesity”. Journal of Clinical Pharmacy and Therapeutics. 47 (2): 184-193. doi:10.1111/jcpt.13574. PMID 34964141. S2CID 245538758.
[3] Amaro A, Sugimoto D, Wharton S (January 2022). “Efficacy and safety of semaglutide for weight management: evidence from the STEP program”. Postgraduate Medicine. 134 (sup1): 5-17. doi:10.1080/00325481.2022.2147326. PMID 36691309. S2CID 256192982.
[4] Doggrell SA (March 2018). “Semaglutide in type 2 diabetes—is it the best glucagon-like peptide 1 receptor agonist (GLP-1R agonist)?” (PDF). Expert Opinion on Drug Metabolism & Toxicology. 14 (3): 371-377. doi:10.1080/17425255.2018.1441286. PMID 29439603. S2CID 3421553. Archived (PDF) from the original on 5 May 2020.
[5] Goldenberg RM, Steen O (March 2019). “Semaglutide: Review and Place in Therapy for Adults With Type 2 Diabetes”. Canadian Journal of Diabetes. 43 (2): 136-145. doi:10.1016/j.jcjd.2018.05.008. PMID 30195966.
[6] Hughes S, Neumiller JJ (January 2020). “Oral Semaglutide”. Clinical Diabetes. 38 (1): 109-111. doi:10.2337/cd19-0079. PMC 6969659. PMID 31975761.
[7] “Ozempic—semaglutide injection, solution”. DailyMed. Archived from the original on 5 Jun. 2021. Retrieved 5 Jun. 2021.
[8] “Rybelsus—oral semaglutide tablet”. DailyMed. Archived from the original on 5 Jun. 2021. Retrieved 5 Jun. 2021.
[9] “Wegovy—semaglutide injection, solution”. DailyMed. 4 Jun. 2021. Archived from the original on 14 Dec. 2021.
[10] “Drug Approval Package: Ozempic (semaglutide) Injection”. U.S. Food and Drug Administration (FDA). 16 Jan. 2018. Archived from the original on 1 Mar. 2021.
[11] “The Top 300 of 2021”. ClinCalc. Archived from the original on 15 Jan. 2024.
[12] “Semaglutide—Drug Usage Statistics”. ClinCalc. Archived from the original on 8 Oct. 2022.
[13] “Wegovy EPAR”. European Medicines Agency. 6 Jan. 2022. Archived from the original on 2 Jul. 2022.
[14] “FDA Approves New Drug Treatment for Chronic Weight Management, First Since 2014”. U.S. Food and Drug Administration (FDA) (Press release). 4 Jun. 2021. Archived from the original on 4 Jun. 2021.
[15] “FDA Approves First Treatment to Reduce Risk of Serious Heart Problems Specifically in Adults with Obesity or Overweight”. U.S. Food and Drug Administration (FDA) (Press release). 8 Mar. 2024. Retrieved 12 Mar. 2024. Public Domain This article incorporates text from this source, which is in the public domain.
[16] Hayashi D, Edwards C, Emond JA, Gilbert-Diamond D, Butt M, Rigby A, et al. (November 2023). Trakada G (ed.). “What Is Food Noise? A Conceptual Model of Food Cue Reactivity”. Nutrients. 15 (22): 4809. doi:10.3390/nu15224809. PMC 10674813. PMID 38004203.
[17] Jarvinen A, Laine MK, Tikkanen R, Castrén ML (2019). “Beneficial Effects of GLP-1 Agonist in a Male With Compulsive Food-Related Behavior Associated With Autism”. Frontiers in Psychiatry. 10: 97. doi:10.3389/fpsyt.2019.00097. PMC 6405420. PMID 30881319.
[18] Kuhn C (25 Sep. 2023). “Patients say drugs like Ozempic help with ‘food noise.’ Here's what that means”. PBS. Archived from the original on 13 Jan. 2024.
[19] Blum D (21 Jun. 2023). “People on Drugs Like Ozempic Say Their ‘Food Noise’ Has Disappeared”. The New York Times. Archived from the original on 11 Jan. 2024.

### SUMMARY

The invention demonstrates several surprising and unexpected advancements in the oral administration of semaglutide, a peptide drug typically vulnerable to enzymatic degradation and harsh gastrointestinal (GI) environments. Unlike conventional dosage forms, this oral thin film (OTF) formulation enables direct buccal or sublingual delivery of semaglutide, thereby bypassing the typical challenges that plague oral peptide therapies, such as poor bioavailability and rapid degradation by gastric enzymes and low pH. The OTF technology stabilizes semaglutide within a carefully designed polymer matrix, maintaining its integrity and activity through both the manufacturing process and passage through the oral cavity. Notably, the physicochemical stability achieved-demonstrated by consistent potency and disintegration properties even after 24 months at ambient conditions—is both unexpected and unprecedented for a biologically sensitive GLP-1 analog peptide.

Additionally, the OTF system achieves robust pharmacokinetic bioequivalence to established oral semaglutide (e.g., Rybelsus®) and subcutaneous formulations, despite the prevailing expectation that large peptides like semaglutide are unsuited for oral absorption due to their size and susceptibility to proteolysis. The film not only protects semaglutide from GI degradation but also ensures rapid and complete disintegration (<60 seconds) for optimal mucosal uptake, minimizing exposure to hostile GI conditions. The synergistic blend of stabilizers, binders, solubilizers, emulsifiers, and carefully engineered disintegration kinetics represents a significant leap over prior art—where such peptides either failed to survive GI transit or required injection. Collectively, these unexpected properties enable a patient-friendly, efficacious, and stable oral peptide therapy, marking a dramatic departure from established limitations in peptide drug delivery.

The present invention provides for an oral thin film (OTF) that includes:

Ingredient

Semaglutide
Water
Binder
Solubilizer
Plasticizer
Emulsifier
Sweetener
Filler
Flavor

The present invention also provides for an oral thin film (OTF) that includes (based on a dry OTF):

| Ingredient | Wt. % of dry strip |
|---|---|
| Semaglutide | 2.6 ± 0.5 |
| Water | 8 ± 2 |
| Carboxymethyl cellulose sodium | 24.1 ± 5 |
| Polyvinylpyrrolidone | 13.8 ± 2.5 |
| Cocoa Butter | 5.6 ± 1 |
| Glycerin | 15.5 ± 3 |
| Polysorbate 80 | 5.2 ± 1 |
| Sucralose | 3 ± 0.5 |
| Microcrystalline cellulose | 9 ± 2 |
| Powder berry flavor | 4.7 ± 1 |
| Liquid berry flavor | 8.6 ± 1.5 |

The present invention also provides for an oral thin film (OTF) that includes (based on a dry OTF):

| Ingredient | Wt. % of dry strip |
|---|---|
| Semaglutide | 2.6 |
| Water | 8 |
| Carboxymethyl cellulose sodium | 24.1 |
| Polyvinylpyrrolidone | 13.8 |
| Cocoa Butter | 5.6 |
| Glycerin | 15.5 |
| Polysorbate 80 | 5.2 |
| Sucralose | 3 |
| Microcrystalline cellulose | 9 |
| Powder berry flavor | 4.7 |
| Liquid berry flavor | 8.6 |

The present invention also provides for an oral thin film (OTF) configured as a unit dose, wherein the unit dose of the oral soluble film includes (based on a dry OTF):

| Ingredient | Mass (mg) in dry strip |
|---|---|
| Semaglutide | 2 |
| Water | 6.4 |
| Carboxymethyl cellulose sodium | 19.3 |
| Polyvinylpyrrolidone | 11 |
| Cocoa Butter | 4.5 |
| Glycerin | 12.4 |
| Polysorbate 80 | 4.1 |
| Sucralose | 2.4 |
| Microcrystalline cellulose | 7.2 |
| Powder berry flavor | 3.8 |
| Liquid berry flavor | 6.9 |

The present invention also provides for an oral thin film (OTF) manufactured from a slurry that includes:

| Ingredient | Mass (g) in 120 g Slurry |
|---|---|
| Semaglutide | 0.72 |
| Water | 94.31 |
| Carboxymethyl cellulose sodium | 6.72 |
| Polyvinylpyrrolidone | 3.84 |
| Cocoa Butter | 1.57 |
| Glycerin | 4.32 |
| Polysorbate 80 | 1.44 |
| Sucralose | 0.84 |
| Microcrystalline cellulose | 2.52 |
| Powder berry flavor | 1.32 |
| Liquid berry flavor | 2.4 |

The present invention also provides for an oral thin film (OTF) manufactured from a slurry that includes:

| Ingredient | Wt. % of slurry |
|---|---|
| Semaglutide | 0.6 ± 0.1 |
| Water | 78.59 ± 10 |
| Carboxymethyl cellulose sodium | 5.6 ± 1 |
| Polyvinylpyrrolidone | 3.2 ± 0.5 |
| Cocoa Butter | 1.31 ± 0.25 |
| Glycerin | 3.6 ± 0.6 |
| Polysorbate 80 | 1.2 ± 0.25 |
| Sucralose | 0.7 ± 0.15 |
| Microcrystalline cellulose | 2.1 ± 0.4 |
| Powder berry flavor | 1.1 ± 0.2 |
| Liquid berry flavor | 2 ± 0.4 |

The present invention also provides for an oral thin film (OTF) manufactured from a slurry that includes:

| Ingredient | Wt. % of slurry |
|---|---|
| Semaglutide | 0.6 |
| Water | 78.59 |
| Carboxymethyl cellulose sodium | 5.6 |
| Polyvinylpyrrolidone | 3.2 |
| Cocoa Butter | 1.31 |
| Glycerin | 3.6 |
| Polysorbate 80 | 1.2 |
| Sucralose | 0.7 |
| Microcrystalline cellulose | 2.1 |
| Powder berry flavor | 1.1 |
| Liquid berry flavor | 2 |

The present invention also provides for the oral administration of an oral thin film (OTF) described herein, as an adjunct to diet and exercise to improve glycemic control in adults with type 2 diabetes mellitus.

The present invention also provides for the oral administration of an oral thin film (OTF) described herein, to reduce the risk of major adverse cardiovascular events in adults with type 2 diabetes mellitus and established cardiovascular disease.

The present invention also provides for the oral administration of an oral thin film (OTF) described herein, to treat type 2 diabetes.

The present invention also provides for the oral administration of an oral thin film (OTF) described herein, to treat obesity.

The present invention also provides for the oral administration of an oral thin film (OTF) described herein, to treat binge eating disorder.

### DETAILED DESCRIPTION

As used herein, “semaglutide” refers to a human GLP-1 receptor agonist (or GLP-1 analog) with a molecular formula C₁₈₇H₂₉₁N₄₅O₅₉, molecular weight 4113.58 g/mol, and structural formula

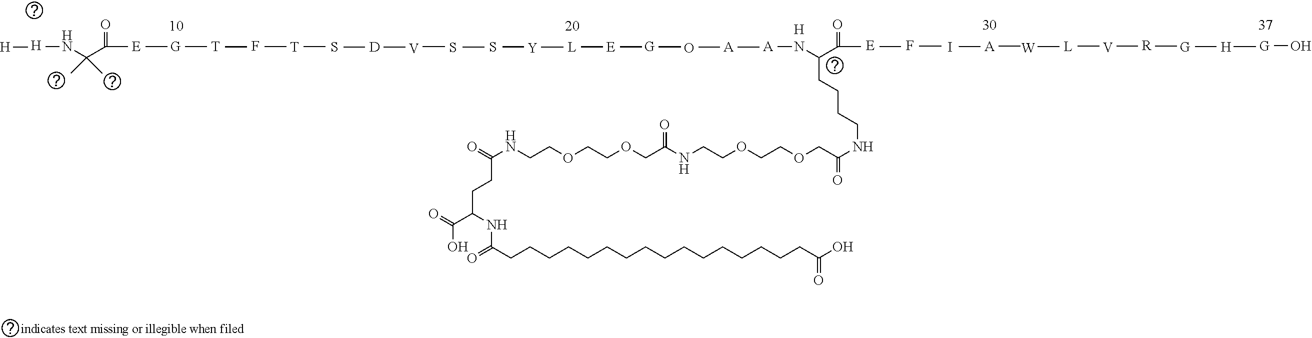

Semaglutide has the IUPAC name 18-[[(1R)-4-[2-[2-[2-[2-[2-[2-[[(5S)-5-[[(2S)-2-[[(2S)-2-[[(2S)-5-amino-2-[[2-[[(2S)-2-[[(2S)-2-[[(2S)-2-[[(2S)-2-[[(2S)-2-[[(2S)-2-[[(2S)-2-[[(2S)-2-[[(2S,3R)-2-[[(2S)-2-[[(2S,3R)-2-[[2-[[(2S)-2-[[2-[[(2S)-2-amino-3-(1H-imidazol-5-yl)propanoyl]amino]-2-methylpropanoyl]amino]-4-carboxybutanoyl]amino]acetyl]amino]-3-hydroxybutanoyl]amino]-3-phenylpropanoyl]amino]-3-hydroxybutanoyl]amino]-3-hydroxypropanoyl]amino]-3-carboxypropanoyl]amino]-3-methylbutanoyl]amino]-3-hydroxypropanoyl]amino]-3-hydroxypropanoyl]amino]-3-(4-hydroxyphenyl)propanoyl]amino]-4-methylpentanoyl]amino]-4-carboxybutanoyl]amino]acetyl]amino]-5-oxopentanoyl]amino]propanoyl]amino]propanoyl]amino]-6-[[(2S)-1-[[(2S)-1-[[(2S,3S)-1-[[(2S)-1-[[(2S)-1-[[(2S)-1-[[(2S)-1-[[(2S)-5-carbamimidamido-1-[[2-[[(2S)-5-carbamimidamido-1-(carboxymethylamino)-1-oxopentan-2-yl]amino]-2-oxoethyl]amino]-1-oxopentan-2-yl]amino]-3-methyl-1-oxobutan-2-yl]amino]-4-methyl-1-oxopentan-2-yl]amino]-3-(1H-indol-3-yl)-1-oxopropan-2-yl]amino]-1-oxopropan-2-yl]amino]-3-methyl-1-oxopentan-2-yl]amino]-1-oxo-3-phenylpropan-2-yl]amino]-4-carboxy-1-oxobutan-2-yl]amino]-6-oxohexyl]amino]-2-oxoethoxy]ethoxy]ethylamino]-2-oxoethoxy]ethoxy]ethylamino]-1-carboxy-4-oxobutyl]amino]-18-oxooctadecanoic acid. Semaglutide has CAS Number 910463-68-2, PubChem CID 56843331, DrugBank DB13928, ChemSpider 34981134, and UNII 53AXN4NNHX.

The peptide backbone is typically produced by yeast fermentation. The main protraction mechanism of semaglutide is albumin binding, facilitated by modification of position 26 lysine with a hydrophilic spacer and a C18 fatty di-acid. Furthermore, semaglutide is modified in position 8 to provide stabilization against degradation by the enzyme dipeptidyl-peptidase 4 (DPP-4). A minor modification was made in position 34 to ensure the attachment of only one fatty di-acid. Semaglutide can exist in the unionized form, or together with a cation, as a pharmaceutically acceptable salt. One suitable pharmaceutically acceptable salt is semaglutide sodium (i.e., the sodium salt of semaglutide) (C₁₈₇H₂₉₁N₄₅O₅₉·Naₓ). Unless expressly indicated otherwise, reference to semaglutide will be to the unionized form.

Semaglutide is a GLP-1 analogue with 94% sequence homology to human GLP-1. Semaglutide acts as a GLP-1 receptor agonist that selectively binds to and activates the GLP-1 receptor, the target for native GLP-1. GLP-1 is a physiological hormone that has multiple actions on glucose, mediated by the GLP-1 receptors. The principal mechanism of protraction resulting in the long half-life of semaglutide is albumin binding, which results in decreased renal clearance and protection from metabolic degradation. Furthermore, semaglutide is stabilized against degradation by the DPP-4 enzyme. Semaglutide reduces blood glucose through a mechanism where it stimulates insulin secretion and lowers glucagon secretion, both in a glucose-dependent manner. Thus, when blood glucose is high, insulin secretion is stimulated, and glucagon secretion is inhibited. The mechanism of blood glucose lowering also involves a minor delay in gastric emptying in the early postprandial phase.

### Physicochemical and Microbiological Stability

The oral thin film (OTF) containing semaglutide is developed to maintain robust physicochemical stability over two years at controlled room temperature (25° C./60% RH). Key attributes to be controlled and maintained for commercial OTF batches include:
pH: Final pH target for solubilized film extract is above 7.0 to maximize semaglutide stability, as peptide degradation accelerates near its isoelectric point (pH˜5.4).
Moisture Content and Water Activity: The OTF is stored in moisture-impermeable packaging and tested at release and annually for water content and a_w, ensuring long-term film integrity and drug release performance.
Microbial Quality: Total aerobic microbial count (TAMC) and total yeast and mold count (TYMC) meet USP <61>/<62> requirements for oral dosage forms (e.g., TAMC<100 CFU/g, TYMC<10 CFU/g, absence of E. coli/Burkholderia cepacia complex), with validated antimicrobial performance via in-process and stability sample testing.
Impurities: Maximum levels of peptide degradation products, excipient-derived breakdown products, and process impurities are defined by validated HPLC and UPLC methods, ensuring specification limits similar to approved semaglutide oral and injectable products.

### Quality Control and Performance Testing

The OTF is subject to comprehensive quality control and performance testing, including but not limited to:
Assay: Content uniformity assessed via HPLC per strip; acceptance is 90-110% of label claim with RSD≤3.0%.
Film Thickness and Weight Variation: Each batch is tested by calibrated micrometer and microbalance to ensure uniform thickness (target: 10-125 μm; ±5%) and mass (target: 80 mg±10%). Variation outside ±10% triggers batch investigation and possible rejection.
Uniformity of Dosage Units: Follows USP <905> for orally disintegrating films.
Mechanical Properties: Tensile strength, folding endurance, and tear resistance measured to ensure robust handling-minimum targets: tensile strength≥5 N/cm², folding endurance≥300 cycles.
Palatability and Organoleptic Properties: Sensory evaluation by panels for taste, texture, mouthfeel, and bitterness masking effectiveness using validated human panels.
Disintegration Time: Measured in simulated saliva at 37° C.; target is complete disintegration<60 seconds in vitro.
Dissolution Testing: Conducted per USP guidance for OTFs using Apparatus I or II; ≥80% of semaglutide released within 15 minutes.
Film Homogeneity: Verified by Near-Infrared (NIR) imaging and Scanning Electron Microscopy (SEM) for uniformity of semaglutide and excipient distribution.
Stability Studies: OTF undergoes ICH stability protocols (zones II/IVb), with interim and 24-month data verifying drug content, potency, disintegration, microbial quality, moisture content, and packaging integrity.

### Bioequivalence and Pharmacokinetics

The OTF is designed and validated against FDA and EMA acceptance criteria for bioequivalence to established oral formulations (e.g., Rybelsus® oral semaglutide):
Pharmacokinetic endpoints: Area under the curve (AUC˜0-24 h˜), maximum plasma concentration (C_max), and time to C_max (T_max) are assessed in crossover studies after dosing healthy volunteers according to the OTF regimen. Bioequivalence is defined if the 90% confidence interval for AUC and C_max ratios falls within 0.800-1.250, meeting FDA/EMA/PMDA regulatory standards.
Safety and Tolerability: Adverse event monitoring in clinical studies confirms a profile matching or improving upon approved oral semaglutide, with particular attention to gastro-intestinal tolerability.
Labeling and Use: Clinical data and in vitro testing establish OTF instructions to be taken on an empty stomach with ≤120 mL water, withholding food and other medications for 30 minutes post administration (as in reference products), and dosing equivalence (e.g., dose escalations of 3 mg, 7 mg, and 14 mg per regimen).

### Embodiments

In specific embodiments, the semaglutide is in the unionized form.

In specific embodiments, the semaglutide exists as a pharmaceutically acceptable salt thereof.

In specific embodiments, the semaglutide exists as semaglutide sodium.

In specific embodiments, the semaglutide exists as a semaglutide conjugate.

In specific embodiments, the semaglutide exists as PEGylated semaglutide.

In specific embodiments, the oral thin film (OTF) includes:

Ingredient

Semaglutide
Water
Binder
Solubilizer
Plasticizer
Emulsifier
Sweetener
Filler
Flavor

In specific embodiments, the oral thin film (OTF) includes (based on a dry OTF):

| Ingredient | Wt. % of dry strip |
|---|---|
| Semaglutide | 2.6 ± 0.5 |
| Water | 8 ± 2 |
| Carboxymethyl cellulose sodium | 24.1 ± 5 |
| Polyvinylpyrrolidone | 13.8 ± 2.5 |
| Cocoa Butter | 5.6 ± 1 |
| Glycerin | 15.5 ± 3 |
| Polysorbate 80 | 5.2 ± 1 |
| Sucralose | 3 ± 0.5 |
| Microcrystalline cellulose | 9 ± 2 |
| Powder berry flavor | 4.7 ± 1 |
| Liquid berry flavor | 8.6 ± 1.5 |

In specific embodiments, the oral thin film (OTF) includes (based on a dry OTF):

| Ingredient | Wt. % of dry strip |
|---|---|
| Semaglutide | 2.6 |
| Water | 8 |
| Carboxymethyl cellulose sodium | 24.1 |
| Polyvinylpyrrolidone | 13.8 |
| Cocoa Butter | 5.6 |
| Glycerin | 15.5 |
| Polysorbate 80 | 5.2 |
| Sucralose | 3 |
| Microcrystalline cellulose | 9 |
| Powder berry flavor | 4.7 |
| Liquid berry flavor | 8.6 |

In specific embodiments, the oral thin film (OTF) is configured as a unit dose, wherein the unit dose of the oral soluble film includes (based on a dry OTF):

| Ingredient | Mass (mg) in dry strip |
|---|---|
| Semaglutide | 2 |
| Water | 6.4 |
| Carboxymethyl cellulose sodium | 19.3 |
| Polyvinylpyrrolidone | 11 |
| Cocoa Butter | 4.5 |
| Glycerin | 12.4 |
| Polysorbate 80 | 4.1 |
| Sucralose | 2.4 |
| Microcrystalline cellulose | 7.2 |
| Powder berry flavor | 3.8 |
| Liquid berry flavor | 6.9 |

In specific embodiments, the oral thin film (OTF) is manufactured from a slurry that includes:

| Ingredient | Mass (g) in 120 g Slurry |
|---|---|
| Semaglutide | 0.72 |
| Water | 94.31 |
| Carboxymethyl cellulose sodium | 6.72 |
| Polyvinylpyrrolidone | 3.84 |
| Cocoa Butter | 1.57 |
| Glycerin | 4.32 |
| Polysorbate 80 | 1.44 |
| Sucralose | 0.84 |
| Microcrystalline cellulose | 2.52 |
| Powder berry flavor | 1.32 |
| Liquid berry flavor | 2.4 |

In specific embodiments, the oral thin film (OTF) is manufactured from a slurry that includes:

| Ingredient | Wt. % of slurry |
|---|---|
| Semaglutide | 0.6 ± 0.1 |
| Water | 78.59 ± 10 |
| Carboxymethyl cellulose sodium | 5.6 ± 1 |
| Polyvinylpyrrolidone | 3.2 ± 0.5 |
| Cocoa Butter | 1.31 ± 0.25 |
| Glycerin | 3.6 ± 0.6 |
| Polysorbate 80 | 1.2 ± 0.25 |
| Sucralose | 0.7 ± 0.15 |
| Microcrystalline cellulose | 2.1 ± 0.4 |
| Powder berry flavor | 1.1 ± 0.2 |
| Liquid berry flavor | 2 ± 0.4 |

In specific embodiments, the oral thin film (OTF) is manufactured from a slurry that includes:

| Ingredient | Wt. % of slurry |
|---|---|
| Semaglutide | 0.6 |
| Water | 78.59 |
| Carboxymethyl cellulose sodium | 5.6 |
| Polyvinylpyrrolidone | 3.2 |
| Cocoa Butter | 1.31 |
| Glycerin | 3.6 |
| Polysorbate 80 | 1.2 |
| Sucralose | 0.7 |
| Microcrystalline cellulose | 2.1 |
| Powder berry flavor | 1.1 |
| Liquid berry flavor | 2 |

In specific embodiments, the oral thin film (OTF) includes semaglutide, water, binder, solubilizer, plasticizer, emulsifier, sweetener, filler, and flavor.

In specific embodiments, the binder includes at least one of pectin, pullulan, hydroxypropyl methylcellulose, and sodium alginate.

In specific embodiments, the binder includes at least one of pectin, pullulan, HPMC, and sodium alginate.

In specific embodiments, the solubilizer includes at least one of polyethylene glycol, MCT oil, and grape seed oil.

In specific embodiments, the solubilizer includes at least one of polyethylene glycol, MCT oil, and grape seed oil.

In specific embodiments, the plasticizer includes at least one of sorbitol, propylene glycol, and polyethylene glycol.

In specific embodiments, the plasticizer includes at least one of sorbitol, propylene glycol, and polyethylene glycol.

In specific embodiments, the emulsifier includes at least one of soy lecithin, propylene glycol, stearic acid, and glyceryl stearate.

In specific embodiments, the emulsifier includes at least one of soy lecithin, propylene glycol, stearic acid, and glyceryl stearate.

In specific embodiments, the sweetener includes at least one of acesulfame potassium, sucrose, stevia, steviol glycosides, and monoammonium glycyrrhizinate.

In specific embodiments, the flavoring agent includes at least one of orange flavor oil, orange flavor powder, spearmint flavor oil, spearmint flavor powder, strawberry flavor oil, and strawberry flavor powder.

In specific embodiments, the oral thin film (OTF) further includes absorption/permeation enhancer(s).

In specific embodiments, the oral thin film (OTF) includes specific polysaccharide and non-polysaccharide binders-such as combining carboxymethylcellulose sodium with pullulan or hydroxypropyl cellulose—for peptide stabilization.

In specific embodiments, the oral thin film (OTF) includes antioxidant(s) such as ascorbic acid, tocopherol, or sodium metabisulfite for protection of semaglutide during manufacture and storage.

In specific embodiments, the oral thin film (OTF) is engineered to achieve water activity below 1.0, for extended stability of peptide APIs, leveraging advanced drying or excipient systems.

In specific embodiments, the oral thin film (OTF) is engineered to achieve water activity below 0.5, for extended stability of peptide APIs, leveraging advanced drying or excipient systems.

In specific embodiments, the oral thin film (OTF) is engineered to achieve water activity below 0.25, for extended stability of peptide APIs, leveraging advanced drying or excipient systems.

In specific embodiments, the oral thin film (OTF) is engineered to achieve water activity below 0.1, for extended stability of peptide APIs, leveraging advanced drying or excipient systems.

In specific embodiments, the oral thin film (OTF) further includes absorption/permeation enhancer(s), such as, e.g., bile salts, surfactants, chitosan, medium-chain fatty acids, etc. to improve transmucosal and/or GI uptake.

In specific embodiments, the oral thin film (OTF) has a mass of 80±16 mg.

In specific embodiments, the oral thin film (OTF) has a mass of 80±8 mg.

In specific embodiments, the oral thin film (OTF) has a mass of 80 mg.

In specific embodiments, the oral thin film (OTF) has the dimensions (length×width×thickness) of 38±3.8 mm×22±2.2 mm×67.5±57.5 m.

In specific embodiments, the oral thin film (OTF) has the dimensions (length×width×thickness) of 38 mm×22 mm×10-125 m.

In specific embodiments, the oral thin film (OTF) is administered to an adult subject.

In specific embodiments, the oral thin film (OTF) is administered to pediatric subject, aged 12 years or older.

In specific embodiments, the oral thin film (OTF) is administered as an adjunct to diet and exercise to improve glycemic control in an adult subject with type 2 diabetes mellitus.

In specific embodiments, the oral thin film (OTF) is administered to reduce the risk of major adverse cardiovascular events in an adult subject with type 2 diabetes mellitus and established cardiovascular disease.

In specific embodiments, the oral thin film (OTF) is administered in combination with a reduced calorie diet and increased physical activity to reduce the risk of major adverse cardiovascular events (cardiovascular death, non-fatal myocardial infarction, or non-fatal stroke) in an adult subject with established cardiovascular disease and either obesity or overweight.

In specific embodiments, the oral thin film (OTF) is administered in combination with a reduced calorie diet and increased physical activity to reduce excess body weight and maintain weight reduction long term in an adult or pediatric subject aged 12 years and older with obesity.

In specific embodiments, the oral thin film (OTF) is administered in combination with a reduced calorie diet and increased physical activity to reduce excess body weight and maintain weight reduction long term in an adult subject with overweight in the presence of at least one weight-related comorbid condition.

In specific embodiments, the oral thin film (OTF) is administered as an antidiabetic medication for the treatment of type 2 diabetes.

In specific embodiments, the oral thin film (OTF) is administered as an anti-obesity medication for long-term weight management.

In specific embodiments, the oral thin film (OTF) is administered as an adjunct to diet and exercise to improve glycemic control in adults with type 2 diabetes.

In specific embodiments, the oral thin film (OTF) is administered as an adjunct to diet and exercise for long-term weight management in adults with obesity (initial body mass index (BMI)≥30 kg/m²) or who are overweight (initial BMI≥27 kg/m²) and have at least one weight-related comorbidity.

In specific embodiments, the oral thin film (OTF) is administered in combination with a reduced calorie diet and increased physical activity, to reduce the risk of cardiovascular death, heart attack, and stroke in obese or overweight adults with cardiovascular disease.

In specific embodiments, the oral thin film (OTF) is administered to treat binge eating disorder (BED), to mitigate obsessive thoughts about food and binging urges.

In specific embodiments, the oral thin film (OTF) contains semaglutide in at least 3 mg.

In specific embodiments, the oral thin film (OTF) contains semaglutide in up to 14 mg.

In specific embodiments, the oral thin film (OTF) contains semaglutide in 3 mg to 14 mg.

In specific embodiments, the oral thin film (OTF) contains semaglutide in 3 mg, 7 mg, or 14 mg.

In specific embodiments, the oral thin film (OTF) is administered to a subject, wherein with each administration one or more oral thin films (OTFs) is administered, such that a total of at least 3 mg of semaglutide is administered to the subject, per administration.

In specific embodiments, the oral thin film (OTF) is administered to a subject, wherein with each administration one or more oral thin films (OTFs) is administered, such that a total of up to 14 mg of semaglutide is administered to the subject, per administration.

In specific embodiments, the oral thin film (OTF) is administered to a subject, wherein with each administration one or more oral thin films (OTFs) is administered, such that a total of 3 mg to 14 mg of semaglutide is administered to the subject, per administration.

In specific embodiments, the oral thin film (OTF) is administered to a subject, wherein with each administration one or more oral thin films (OTFs) is administered, such that a total of 3 mg, 7 mg, or 14 mg of semaglutide is administered to the subject, per administration.

In specific embodiments, multiple oral thin films (OTFs) a day are independently administered to the subject.

In specific embodiments, 2-10 oral thin films (OTFs) a day are independently administered to the subject.

In specific embodiments, the oral thin film (OTF) is administered at least 30 minutes before the first food, beverage, or other oral medications of the day with no more than 4 ounces of plain water only.

In specific embodiments, the oral thin film (OTF) is administered with the following dosing regimen: start with 3 mg semaglutide once daily for 30 days, after 30 days on the 3 mg dose, increase the dose to 7 mg once daily.

In specific embodiments, the oral thin film (OTF) is administered with the following dosing regimen: dose may be increased to 14 mg once daily if additional glycemic control is needed after at least 30 days on the 7 mg dose.

In specific embodiments, the oral thin film (OTF) is administered once weekly as an adjunct to diet and increased physical activity, on the same day each week, at any time of day, with or without meals.

In specific embodiments, the oral thin film (OTF) is administered with the following dosing regimen: initiate at 0.25 mg once weekly for 4 weeks; then follow the dosage escalation schedule, titrating every 4 weeks to achieve the maintenance dosage.

In specific embodiments, the oral thin film (OTF) is administered such that the maintenance dosage of semaglutide in adults is either 2.4 mg (recommended) or 1.7 mg once weekly.

In specific embodiments, the oral thin film (OTF) is administered such that the maintenance dosage of semaglutide in pediatric patients aged 12 years and older is 2.4 mg once weekly.

In specific embodiments, the oral thin film (OTF) is administered at least 15 minutes before food/beverage/other medication.

In specific embodiments, the oral thin film (OTF) is administered at least 30 minutes before food/beverage/other medication.

In specific embodiments, the oral thin film (OTF) is administered at least 45 minutes before food/beverage/other medication.

In specific embodiments, the oral thin film (OTF) is administered with not more than 100 mL water.

In specific embodiments, the oral thin film (OTF) is administered with not more than 120 mL water.

In specific embodiments, the oral thin film (OTF) is administered with not more than 150 mL water.

In specific embodiments, the oral thin film (OTF) is administered with not more than 300 mL water.

In specific embodiments, the oral thin film (OTF) is provided in resealable or single-use moisture-impermeable composite pouches, with desiccant-integrated primary packaging.

In specific embodiments, the oral thin film (OTF) is provided in a container-closure systems validated as both child-resistant and senior-accessible, with physical dimensions facilitating daily home use or discrete dosing.

In specific embodiments, the oral thin film (OTF) is individually-sealed.

In specific embodiments, the oral thin film (OTF) is individually-sealed, with multi-strip blisters, child-resistant or moisture-impermeable pouches, primary containers passing USP <661> leachables/extractables and humidity barrier testing.

In specific embodiments, the oral thin film (OTF) has microbial limits in compliance with USP <61>/<62>.

In specific embodiments, the oral thin film (OTF) has microbial limits in compliance with USP <61>/<62> (e.g., TAMC<100 CFU/g, TYMC<10 CFU/g, absence of E. coli/Burkholderia cepacia).

In specific embodiments, the oral thin film (OTF) has impurity limits, e.g., peptide degradation products, total unspecified impurities not more than 1.0%.

In specific embodiments, the oral thin film (OTF) has impurity limits, e.g., peptide degradation products, total unspecified impurities not more than 0.5%.

In specific embodiments, the oral thin film (OTF) maintains 90% semaglutide content after 24 months at 25° C./60% RH.

In specific embodiments, the oral thin film (OTF) maintains≥95% semaglutide content after 24 months at 25° C./60% RH.

In specific embodiments, the oral thin film (OTF) maintains≥97.5% semaglutide content after 24 months at 25° C./60% RH.

In specific embodiments, the oral thin film (OTF) maintains 99% semaglutide content after 24 months at 25° C./60% RH.

### EXAMPLES

### Example 1: Formulation 1, Slurry and Oral Thin Film (OTF)

| | | | | | SLURRY | | | OTF | | |
|---|---|---|---|---|---|---|---|---|---|---|
| | | | | | Mass | | | Mass | | |
| | | | | | (g) in | Wt. % | | (mg) | Wt. % | |
| | | | | | 120 g | of | CALCU- | in dry | of dry | CALCU- |
| Ingredient | Vendor | CAS# | Function(s) | Alternative(s) | Slurry | slurry | LATED | strip | strip | LATED |
| Semaglutide | | 910463-68-2 | API | N/A | 0.72 | 0.6 | 0.60 | 2 | 2.6 | 2.50 |
| Water | | 7732-18-5 | Solvent | N/A | 94.31 | 78.59 | 78.59 | 6.4 | 8 | 8.00 |
| Carboxymethyl | CP Kelco | 9004-32-4 | Binder | Pectin, Pullulan, | 6.72 | 5.6 | 5.60 | 19.3 | 24.1 | 24.13 |
| cellulose sodium | | | | Hydroxypropyl | | | | | | |
| | | | | methylcellulose, | | | | | | |
| | | | | Sodium alginate | | | | | | |
| Polyvinyl- | Mutchler Inc | 9003-39-8 | Binder | Pectin, Pullulan, | 3.84 | 3.2 | 3.20 | 11 | 13.8 | 13.75 |
| pyrrolidone | | | | Hydroxypropyl | | | | | | |
| | | | | methylcellulose, | | | | | | |
| | | | | Sodium alginate | | | | | | |
| Cocoa Butter | Jedwards | 8002-31-1 | Solubilizer | Polyethylene glycol, | 1.57 | 1.31 | 1.31 | 4.5 | 5.6 | 5.63 |
| | International | | | MCT oil, Grape | | | | | | |
| | Inc. | | | seed oil | | | | | | |
| Glycerin | Hawkins Inc | 56-81-5 | Plasticizer | Sorbitol, Propylene | 4.32 | 3.6 | 3.60 | 12.4 | 15.5 | 15.50 |
| | | | | glycol, Polyethylene | | | | | | |
| | | | | glycol | | | | | | |
| Polysorbate 80 | Spectrum | 9005-65-6 | Emulsifier | Soy Lecithin, | 1.44 | 1.2 | 1.20 | 4.1 | 5.2 | 5.13 |
| | | | | Propylene glycol, | | | | | | |
| | | | | Stearic acid, | | | | | | |
| | | | | Glyceryl stearate | | | | | | |
| Sucralose | JK Sucralose | 56038-13-2 | Sweetener | Acesulfame potassium, | 0.84 | 0.7 | 0.70 | 2.4 | 3 | 3.00 |
| | Inc | | | Sucrose, Stevia, | | | | | | |
| | | | | Steviol glycosides, | | | | | | |
| | | | | Monoammonium | | | | | | |
| | | | | glycyrrhizinate | | | | | | |
| Microcrystalline | FMC | 9004-34-6 | Filler | Starch, maltodextrin, | 2.52 | 2.1 | 2.10 | 7.2 | 9 | 9.00 |
| cellulose | BioPolymer | | | amylose, hydroxypropyl | | | | | | |
| | | | | cellulose | | | | | | |
| Powder berry | Gold Coast | | Flavor | Orange flavor oil, | 1.32 | 1.1 | 1.10 | 3.8 | 4.7 | 4.75 |
| flavor | | | | Orange flavor powder, | | | | | | |
| | | | | Spearmint flavor oil, | | | | | | |
| | | | | Spearmint flavor powder, | | | | | | |
| | | | | Strawberry flavor oil, | | | | | | |
| | | | | Strawberry flavor powder | | | | | | |
| Liquid berry | Gold Coast | | Flavor | Orange flavor oil, | 2.4 | 2 | 2.00 | 6.9 | 8.6 | 8.63 |
| flavor | | | | Orange flavor powder, | | | | | | |
| | | | | Spearmint flavor oil, | | | | | | |
| | | | | Spearmint flavor powder, | | | | | | |
| | | | | Strawberry flavor oil, | | | | | | |
| | | | | Strawberry flavor powder | | | | | | |
| | | | TOTAL | | 120 | | 100.00 | 80 | | 100.00 |

### Example 2: Method of Manufacturing Oral Thin Film (OTF)

Weigh out the following ingredients in the specified amounts:
94.31 g of room temperature purified water
6.72 g carboxymethyl cellulose sodium
3.84 g polyvinylpyrrolidone
1.57 g cocoa butter
4.32 g glycerin
1.44 g polysorbate 80
0.84 g sucralose
2.52 g microcrystalline cellulose
1.32 g berry flavor powder
2.40 g berry flavor liquid
0.72 g semaglutide

Heat the cocoa butter to 95 F to liquify the solid.

Using a benchtop hand blender, blend the water and add the ingredients in the following order. Allow 20 seconds between each ingredient:
1. Glycerin
2. Cocoa butter
3. Polysorbate 80
4. Sucralose
5. Berry powder
6. Berry liquid
7. Microcrystalline cellulose
8. Semaglutide
9. Polyvinylpyrrolidone
10. Carboxymethyl cellulose sodium

Blend the solution for 5 minutes after adding the last ingredient.

Using a Gardco film applicator set at 700 mcm, extrude the solution onto siliconized paper. Place the extruded solution in a convection oven. Dry the product using no heat, only the fan for 50-70 minutes. Remove the product from the oven. Using a straight edge and a blade, cut the product into 38×22 mm strips.

### Batch Formulation and Production

### Example 3: Semaglutide Oral Thin Film, 7 mg Dose-Batch Formulation

| Item | | | mg/strip | Wt. % |
|---|---|---|---|---|
| No. | Component | Grade | (80 mg) | (of dry) |
| 1 | Semaglutide | API/USP | 7.0 | 8.8 |
| 2 | Carboxymethylcellulose | NF/USP | 19.3 | 24.1 |
| | Na | | | |
| 3 | Polyvinylpyrrolidone | NF/USP | 11.0 | 13.8 |
| 4 | Cocoa butter | Food/Ph. Grd | 4.5 | 5.6 |
| 5 | Glycerin | USP | 12.4 | 15.5 |
| 6 | Polysorbate 80 | NF/USP | 4.1 | 5.2 |
| 7 | Sucralose | USP | 2.4 | 3.0 |
| 8 | Microcrystalline | USP/Ph. Grd | 7.2 | 9.0 |
| | cellulose | | | |
| 9 | Berry flavor powder | Food Grd | 3.8 | 4.7 |
| 10 | Berry flavor liquid | Food Grd | 6.9 | 8.6 |

All excipients are compendial or food grade, and comply with USP/NF and FDA requirements for oral films. The process includes blending, casting, and controlled drying steps following a validated SOP, with strict batch record-keeping and yield reconciliation.

### Regulatory, Packaging, and Storage Guidelines

Packaging: OTF is sealed in individually moisture-impermeable foil sachets. Container-closure systems are validated to maintain product ID, quality, and deliverable dose. All primary packaging passes USP <661> testing for extractables/leachables and moisture barrier integrity.
Labeling: Dosing instructions, stability, storage (25° C.; protect from humidity/light), warnings, and pediatric/adult indications are compliant with current FDA/EMA/ICH requirements.
Shelf Life: Retest intervals and expiration dating are justified by real-time, accelerated, and worst-case stability data, with minimum shelf life proposed as 24 months post manufacture.

1. An oral thin film (OTF) comprising:
semaglutide present in 2.6 wt. %;
water present in 8 wt. %;
carboxymethyl cellulose sodium present in 24.1 wt. %;
polyvinylpyrrolidone present in 13.8 wt. %;
cocoa butter present in 5.6 wt. %;
glycerin present in 15.5 wt. %;
polysorbate 80 present in 5.2 wt. %;
sucralose present in 3 wt. %;
microcrystalline cellulose present in 9 wt. %;
powder berry flavor present in 4.7 wt. %; and
liquid berry flavor present in 8.6 wt. %.

2. The oral thin film (OTF) of claim 1, exhibiting≤10% strip-to-strip content variation and in vitro disintegration time<60 s.

3. The oral thin film (OTF) of claim 1, that maintains>90% semaglutide content and complete disintegration after 24 months at 25° C./60% RH.

4. The oral thin film (OTF) of claim 1, having tensile strength≥5 N/cm², folding endurance≥300 cycles, and microbial limits as per USP <61>/<62> for oral dosage forms.

5. A method comprising administering the oral thin film (OTF) of claim 1 to a subject.
